(19) United States
(12) Patent Application Publication
(10) Pub. No.: US 2025/0339136 A1
(43) Pub. Date: Nov. 6, 2025

(54) MEDICAL DEVICES, SYSTEMS, AND METHODS FOR PROVIDING TRACTION TO TISSUE

(71) Applicant: Boston Scientific Scimed, Inc., Maple Grove, MN (US)

(72) Inventors: Mauro Víquez Gonzalez, San Isidro (CR); Marco Vinicio ARIAS ALPIZAR, San José (CR); Gonzalo Jose SAENZ VILLALOBOS, Alajuela (CR); Fernando VALERIO GUERRERO, Alajuela (CR); Oscar Andres SEGURA ELIZONDO, San Jose (CR); Viviana ARTAVIA SALAS, Heredia (CR); Erick Gerardo VAN PATTEN RIVERA, San Jose (CR); Gustavo CARVAJAL BONILLA, Heredia (CR); Valeria ZUÑIGA VARGAS, Austin, TX (US); Rafael BERENZON, Cartago (CR); Gabriela GAMBOA BASTOS, San Jose (CR); Esteban de la Trinidad JIMENEZ SANCHEZ, San Jose (CR)

(73) Assignee: Boston Scientific Scimed, Inc., Maple Grove, MN (US)

(21) Appl. No.: 19/194,407

(22) Filed: Apr. 30, 2025

Related U.S. Application Data
(60) Provisional application No. 63/640,906, filed on May 1, 2024.

(51) Int. Cl. A61B 17/02

(52) U.S. Cl. CPC A61B 17/0218

(57) ABSTRACT

Disclosed are medical devices. In an aspect, a medical device of the disclosed medical devices may comprise a traction hook configured for grasping tissue. The medical device may further comprise an anchor. In some aspects, the medical device may additionally comprise a member connecting the anchor to the traction hook.

### CROSS-REFERENCE TO RELATED APPLICATIONS

This application claims the benefit of priority to U.S. Provisional Application No. 63/640,906, filed on May 1, 2024, which is incorporated by reference herein in its entirety.

### TECHNICAL FIELD

Various aspects of this disclosure generally relate to medical devices and systems and for providing traction to tissue and related methods. In particular, aspects of this disclosure relate to medical devices, systems, and related methods for devices that apply traction to tissue to facilitate resection.

### BACKGROUND

Endoscopic procedures may involve removing a lesion from a body lumen. For example, endoscopic submucosal dissection (ESD) utilizes endoscopic tools to remove cancerous or other lesions from the gastrointestinal tract. For example, an instrument, such as a surgical knife, may be used via a working channel of an endoscope in order to cut and remove the lesion. In some procedures, a lifting agent may be injected under the lesion prior to removing the lesion, so as to facilitate access by the surgical knife or other instrument. A need exists for applying traction or tension to a lesion or other portion of a body lumen to facilitate medical procedures, such as ESD.

### SUMMARY

Each of the aspects disclosed herein may include one or more of the features described in connection with any of the other disclosed aspects.

In an example, a medical device comprises a traction hook configured for grasping tissue, an anchor, and a member connecting the anchor to the traction hook.

Any of the aspects disclosed herein may include any of the following features, alone or in combination. The member may be configured to be coupled to an external surface of a shaft of an insertion device by one or more attachment mechanisms. The medical device may comprise a ring attached to the anchor. The member may extend through the ring. The member may be movable relative to the ring and fixedly coupled to the anchor. The anchor may include an anchor hook. The member may be retractable. The anchor may include a body defining a hollow interior. The member may be retractable into the hollow interior of the anchor. The member may be biased into a retracted position inside the hollow interior of the anchor. The anchor may include a corkscrew. The anchor may include a weighted ball. The member may be a first member, and the medical device may include a handle having a first actuator configured to deploy the traction hook, a second actuator configured to adjust a tension of the first member, a third actuator configured to deploy the anchor, and a fourth actuator configured to adjust a tension of a second member coupled to the anchor. The first member and the second member may be coupled to one another by a capsule in a deployed configuration of the medical device. The handle may comprise a fifth actuator configured to secure the first member to the second member and thus secure a tension of the first member and the second member. The handle may comprise a sixth actuator configured to separate the first member and the second member from the handle.

In another aspect, a medical device may comprise: a handle including a plurality of first actuators, a second actuator, and a third actuator, a sheath, a plurality of members extending through the sheath, and a plurality of anchoring mechanisms, each anchoring mechanism at a distal end of a respective member of the plurality of members. Each of the plurality of first actuators may be configured to deploy at least one anchoring mechanism of the plurality of anchoring mechanisms. The second actuator may be configured to secure the plurality of members to one another. The third actuator may be configured to separate the plurality of members from the handle and the sheath.

Any of the aspects disclosed herein may include any of the following features, alone or in combination. The handle may comprise a plurality of fourth actuators configured to adjust a tension of a member of the plurality of members. Each of the plurality of first actuators may be configured to deploy the at least one anchoring mechanism independently of others of the plurality of anchoring mechanisms.

In another aspect, a medical method may comprise: attaching a traction hook to the target tissue, and placing an anchor proximate to the traction hook. The anchor may be connected to the traction hook via a cable, wire, or thread.

Any of the aspects disclosed herein may include any of the following features, alone or in combination. Placing the anchor proximate to the traction hook may comprise attaching the anchor to a body lumen.

It may be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts aspects of an exemplary device for applying traction to a tissue.

FIG. 2 depicts aspects of another exemplary system for applying traction to a tissue.

FIG. 3 depicts aspects of another exemplary system for applying traction to a tissue.

FIG. 4A depicts a perspective view of a device for applying traction to a tissue.

FIG. 4B depicts the device of FIG. 4A applying traction to a tissue.

FIG. 5 depicts aspects of another exemplary device for applying traction to a tissue.

FIG. 6 depicts aspects of another exemplary device for applying traction to a tissue.

FIG. 7A depicts aspects of another exemplary device for applying traction to a tissue.

FIG. 7B depicts a cut-away view of the exemplary device of FIG. 7A.

FIG. 7C depicts a perspective view of a portion of the exemplary device of FIG. 7A.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Disclosed are systems, devices, and methods for applying traction to tissue. During an endoscopic procedure, one or more accessory devices may be used along with a medical device (e.g., an endoscope) in order to perform a medical procedure such, as, for example, tissue resection. In order to facilitate resection, the disclosed systems and/or devices may apply traction to a target tissue to be resected or to another tissue (e.g., a tissue adjacent to a tissue to be resected). The traction may facilitate tissue resection by pulling the target tissue away from adjacent walls of a body lumen. As the target tissue is being resected, the disclosed systems and/or devices may continue to apply traction to the target tissue, to lift away the tissue and move it out of the way to provide for increased visualization using a camera of the medical device.

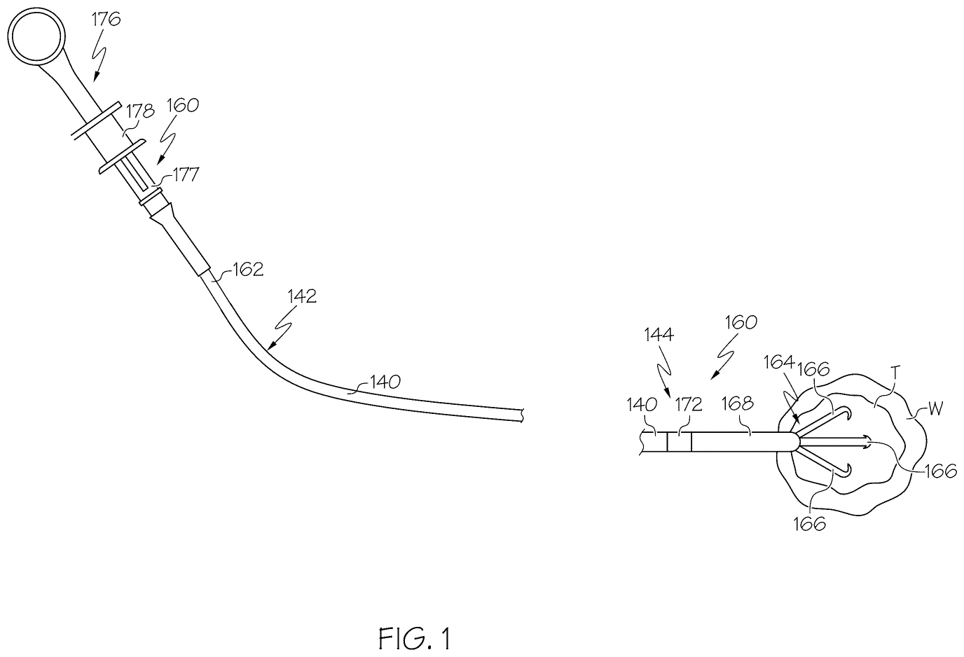

FIG. 1 depicts an exemplary medical device 160. As described herein, medical device 160 may include an end effector 164 that may apply traction and/or tension to a target tissue (e.g., to a lesion or to an area neighboring a lesion) and/or may create a tissue flap. In examples, medical device 160 may be inserted into an endoscope or any other insertion device (e.g., scope or sheath) known in the art. For example, medical device 160 may be delivered to a treatment site through a working channel of the insertion device. Although endoscopes may be referenced herein, the disclosure also encompasses other types of devices, such as duodenoscopes, bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to endoscopes herein should be understood to encompass any of the above medical devices or any other similar medical device.

The medical device 160 may include a handle 176 and a shaft 140. The shaft 140 may extend from a proximal end 142 to a distal end 144. The medical device 160 may also include an end effector 164 at a distalmost end of distal end 144 of shaft 140. End effector 164 may include three arms 166, which may be partially contained within a capsule 168. The end effector 164 may be removably coupled to shaft 140 via a bushing 172.

In some examples, arms 166 may have a tripod shape. For example, arms 166 may have the same length and be separated from one another by the same angles. In alternatives, arms 166 may have different lengths and different angular separations. The arms 166 may be designed with for example, gripping elements that avoid causing damage to the surrounding tissue. In another embodiment, the gripping elements may be distributed over an increased surface area of the gripping elements to distribute the gripping force over a larger area of tissue. In some embodiments, there may be more than three arms 166. The arms are configured to grasp a target tissue T.

The end effector 164, capsule 168 and arms 166 may be configured to be separated from bushing 172 when end effector 164 is deployed. In other words, end effector 164 may be separated from shaft 140 during deployment, such that shaft 140 may be removed from a body lumen (e.g., removed proximally through a working channel of an insertion device, such as an endoscope), while end effector 164 remains in the body lumen. The capsule 168 may include any elements known in the art that help to deploy end effector 164 (e.g., any features of any clip devices known in the art, such as yoke(s), clevis(es), frangible pin(s), and the like).

A handle 176 may be disposed at a proximal end of shaft 140. Handle 176 may include a handle body 177 and one or more actuators 178 for controlling aspects of end effector 164. For example, actuator 178 may be a spool that moves longitudinally along handle body 177. Handle 176 may have any of the features of any medical device handle known in the art. Handle 176 may include additional and/or alternative actuators. A configuration of handle 176 is merely exemplary and shown for illustrative purposes. Movement of actuator 178 relative to handle body 177 may open and/or close arms 166 and/or deploy end effector 164 (separate end effector 164 from shaft 140). An operation of medical device 160 and further features of medical device 160 are described below.

A method of using medical device 160 will now be described. End effector 164 and shaft 140 may be inserted into a body lumen of a subject (e.g., by inserting end effector 164 and shaft 140 into a port of an insertion device and passing them through a working channel of the insertion device). Alternatively, medical device 160 may be inserted separately from or external to an insertion device (e.g., as an over-the-scope device).

Once end effector 164/shaft 140 is positioned at a target site, a user may deploy end effector 164 to the target site, thereby separating end effector 164 from shaft 140. End effector 164 may be deployed using any method/approach that is typically used to deploy a clip. For example, an operator may move actuator 178 proximally to .first close arms 166 and then to separate capsule 168 from bushing 172. Thus, end effector 164 may be separated/severed from shaft 140 and bushing 172. However, the mechanics described above are merely exemplary, and medical device may be operated using any mechanisms known in the art.

End effector 164 may be actuated using actuator 178, for example, to grab onto a target tissue T by closing arms 166 to pull target tissue T away from a surrounding (e.g., adjacent tissue wall W of the body lumen. For example, all of arms 166 may be closed simultaneously. Target tissue T may be a lesion or ulcer, or other tissue requiring resection or treatment. Target tissue T may be disposed on tissue wall W of the body lumen. An imaging device (e.g., camera) of an insertion device may be used to guide end effector 164 to target tissue T. Once end effector 164 has pulled target tissue T away from tissue wall W of the body lumen, the operator may use another medical device (e.g., a knife) to resect or otherwise treat target tissue T.

During resection of target tissue T, end effector 164 may continue to grasp and apply traction to target tissue T, forming a tissue flap and keeping the target tissue positioned to facilitate resection. Another medical instrument (e.g., a basket) may be used to retrieve target tissue T and end effector 164 and to remove target tissue T from the subject's body. As compared with conventional clip devices, medical device may provide for increased traction on any type of damaged tissue or area.

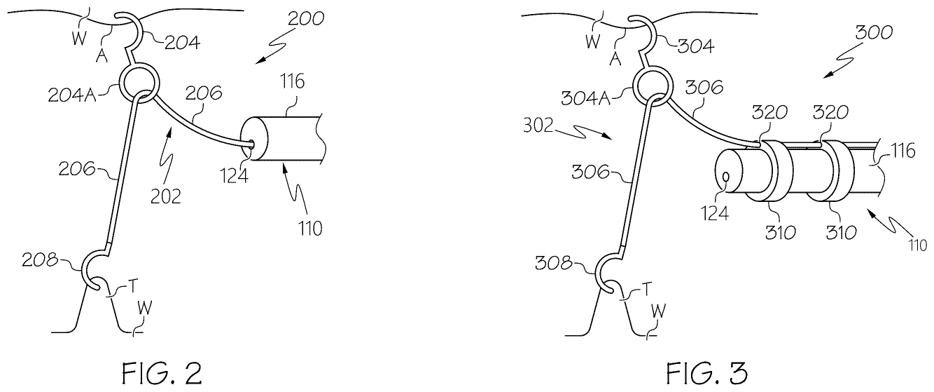

FIG. 2 depicts aspects of a system 200 for applying traction to a tissue. System 200 may include an insertion device, such as an endoscope 110 having a shaft 116. Endoscope 110 may also be used with device 160, described above. The medical system 200 may also include a traction device 202 that includes an anchor hook 204 (a first hook) and a traction hook 208 (a second hook).

A cable 206 (or wire, suture, thread, or other similar member) may extend between anchor hook 204 and traction hook 208. For example, anchor hook 204 may be connected to (or formed integrally with) an anchor ring 204A. Cable 206 may extend through anchor ring 204A and may be movable relative to anchor ring 204A. Cable 206 may be fixedly coupled to a proximal end of traction hook 208. In alternatives, cable 206 may be integrally formed with traction hook 208. Other arrangements of cable 206 are contemplated. For example, cable 206 may include multiple cable pieces or elements that are coupled to one another. For example, a first cable piece may be coupled to anchor hook 204 (e.g., via anchor ring 204A) and a second cable piece may be coupled to traction hook 208 (e.g., via a ring similar to anchor ring 204A or any suitable structure). The first cable piece and the second cable piece may be coupled to one another.

Shaft 116 may be navigated to target tissue T on tissue wall W of a body lumen of a patient. The traction device may be extended through working channel 124 of endoscope 110 and may extend distally outward of shaft 116. For example, cable 206 may extend through working channel 124. When positioned near target tissue T, an operator may use a grasper (not shown and having features of any grasper known in the art) or another instrument to connect traction hook 208 to target tissue T, and to connect anchor hook 204 to a portion of tissue wall W, which may be referred to as an anchor portion A of tissue wall W.

In some examples, anchor portion A may be positioned a portion of tissue wall W opposite target tissue T. With traction hook 208 in position attached to target tissue T, and anchor hook 204 connected to anchor portion A of tissue wall W opposite target tissue T, cable 206 may be actuated via an actuator (e.g., an having any of the properties of actuator 178 of handle 176 in FIG. 1), to move cable 206 in a proximal direction, making cable 206 taut and pulling target tissue T away from tissue wall W. Ring 204A may function similar to a pulley, and cable 206 may move relative to and through ring 204A. Thereafter, as discussed above, another medical device (e.g., a knife) may be used to resect or otherwise treat target tissue T. Cable 206 may be sized and shaped such that other medical devices (e.g., graspers or knives) may extend through working channel 124 along with cable 206.

FIG. 3 depicts aspects of another exemplary system 300 for applying traction to a tissue, similar to system 200. Unless otherwise specified herein, system 300 may have any of the features of system 200. Similar to medical system 200, medical system 300 may include a traction device 302 that includes an anchor hook 304 (a first hook, having any of the properties of anchor hook 204) and a traction hook 308 (a second hook, having any of the properties of traction hook 208).

However, traction device 302 may include a cable 306 (having any of the properties of cable 206 except as specified herein) that extends outside of shaft 116 of endoscope 110 instead of through working channel 124. Thus, traction device 302 may be an over-the-scope device. For example, a plurality of attachment members 310 (e.g., rings) may be coupled to (e.g., extend around) shaft 116. Each of attachment members 310 may include a lumen 320 through which cable 306 extends. Any suitable number of attachment members 310 may be utilized (e.g., one or more).

Similarly to system 200, shaft 116 may be navigated to target tissue T on tissue wall W of a body lumen of a patient. When positioned near target tissue T, an operator may use a grasper (not shown) to connect traction hook 308 to target tissue T, and to connect anchor hook 304 to a portion of tissue wall W, which may be referred to as an anchor portion A of tissue wall W. For example, the grasper may be extended through working channel 124.

In some examples, anchor portion A may be positioned a portion of tissue wall W opposite target tissue T. Similarly to traction device 202, cable 306 may extend through an anchor ring 304A and be coupled to traction hook 308. With traction hook 308 in position attached to target tissue T, and anchor hook 304 connected to anchor portion A of tissue wall W opposite target tissue T, cable 306 may be actuated via an actuator (e.g., actuator 178 on handle 176 in FIG. 1), to move in a proximal direction, making cable 306 taut and pulling target tissue T away from tissue wall W. Anchor ring 304A may function similar to a pulley.

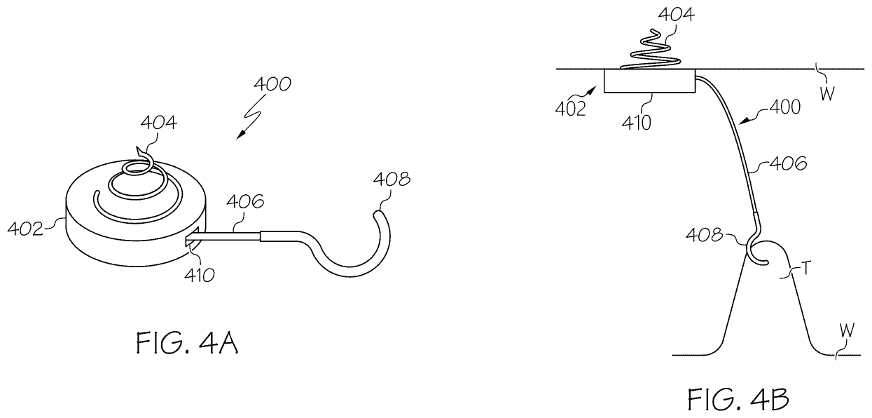

FIGS. 4A and 4B depicts another device 400 for applying traction to a tissue. Device 400 includes an anchor device 402 and a traction hook 408 connected to each other by retractable cable 406. Traction hook 408 may have any of the properties of hooks 208, 304, 308 discussed above. Cable 406 may have any of the properties of cables 206 and 306. Although the term cable is used herein, cable 406 may include a thread, string, suture, wire, or other similar member.

Anchor device 402 includes an anchor attachment portion 404 and a body 410. For example, body 410 may have (e.g., define) a hollow interior cavity for housing portions of retractable cable 406 (including in a retracted configuration of device 400). Body 410 may include a biasing member (e.g., a spring or other element), such that retractable cable 406 is biased to a configuration in which at least a portion of cable 406 is retracted within body 410. In some examples, as shown in FIG. 4A, device 400 may be biased to a configuration in which nearly an entirety (or an entirety) of cable 406 is within body 410, such that, in the neutral configuration, traction hook 408 is adjacent to body 410. In other examples, device 400 may be biased to a configuration in which a predefined length of cable 406 is outside of body 410, such that traction hook 408 is separated from body 410 by the predetermined length.

Attachment portion 404 may extend from a side surface of body 410. As shown in FIG. 4A, attachment portion 404 may include a corkscrew wire that may be rotatable in order to secure anchor device 402 to a body tissue. Alternatively, attachment portion 404 may include one or more hooks, barbs, adhesives, or other elements for coupling anchor device 402 to body tissue.

An operator may use a grasper (not shown) or other accessory device and an insertion device (e.g., endoscope) to position device 400 within a body lumen of a subject adjacent to a target tissue T on tissue wall W. As shown in FIG. 4B, the operator may use the grasper or other device to attach traction hook 408 to target tissue T and use attachment portion 404 to attach anchor device 402 to tissue wall W (e.g., at a position opposite target tissue T). Retractable cable 406 may be biased to a retracted configuration, as described above. Thus, retractable cable 406 is taut in the configuration shown in FIG. 4B. As such, the tension applied by retractable cable 406 pulls target tissue T away from tissue wall W. As discussed above, a knife or other device may be used to resect or otherwise treat target tissue T.

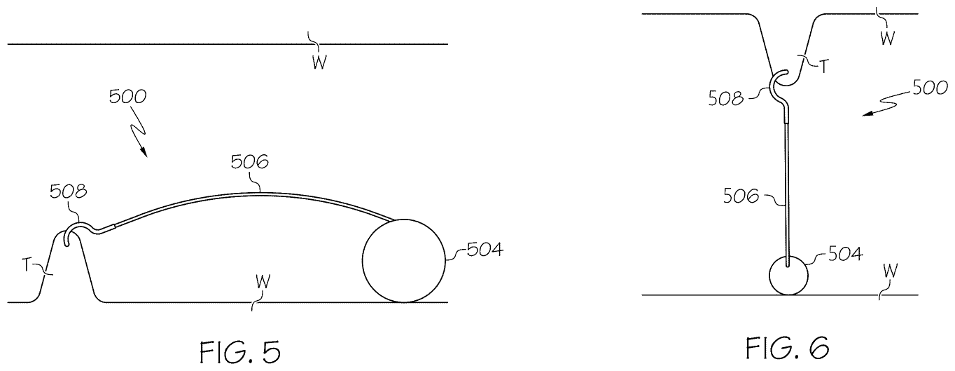

FIGS. 5 and 6 depicts aspects of another exemplary device 500 for applying traction to a tissue. Device 500 includes an anchor body 504 and a traction hook 508 connected to each other by cable 506. Cable 506 may have any of the features of any of the cables disclosed herein. Although the term “cable” is used herein, cable 506 may be any suitable member, including, for example, a thread, suture, wire, etc. Anchor body 504 may include a weighted object or body, such as a pellet or ball.

An operator may use a grasper (or another device) and/or an insertion device (not shown) to transport device 500 within a body lumen to a target tissue T on tissue wall W. The operator may use the grasper to attach traction hook 508 to target tissue T and position anchor body 504 at a position within the body lumen in the proximity of target tissue T. Anchor body 504 may be placed by the operator such that cable 506 is taut and provides tension, thus pulling target tissue T away from tissue wall W.

FIG. 6 depicts another exemplary arrangement of device 500 in a body lumen. An operator may use the grasper (or other device) to attach traction hook 508 to target tissue T and position anchor body 504 at a position within the body lumen in the proximity of target tissue T on an opposite side of the body lumen, below target tissue T. Anchor body 504 may then be pulled by the force of gravity to help pull cable 506 taut, providing tension and pulling target tissue T away from tissue wall W.

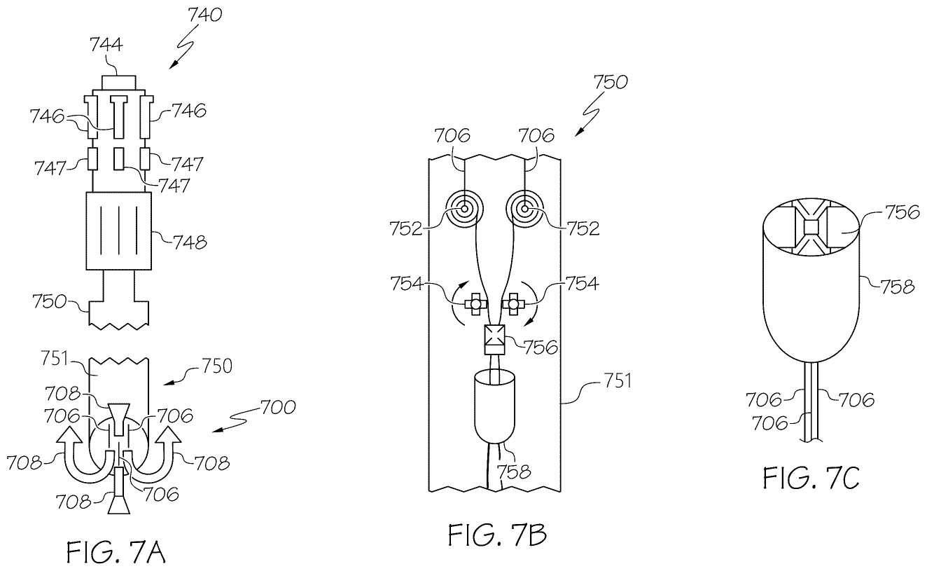

FIGS. 7A-7C depict aspects of another exemplary device 700 for applying traction to a tissue. Device 700 includes a proximal handle portion 740 and a distal insertion portion 750 to be inserted into a body lumen of a patient. For example, insertion portion 750 may be extended through a working channel of an insertion device (e.g., working channel 124 of endoscope 110).

Insertion portion 750 includes a sheath 751. FIG. 7B shows internal components of sheath 751. A plurality of cables 706 may extend through sheath 751. A hook 708 or other anchoring mechanism (e.g., barb, corkscrew, suture, staple, tag, etc.) may be connected to a distal end each of cables 706. For example, each of hooks 708 may extend radially outward from a central longitudinal axis of sheath 751 when they are received within sheath 751. Although the term “cable” is used herein, it will be appreciated that cables 706 may be, for example, suturing strings, wires, threads, or similar members.

Handle portion 740 includes a variety of actuators, including a button 744, one or levers or sliders 746, one or more tension knobs 747, and a securing knob 748. As shown in FIG. 7A, button 744 may be on a proximalmost end of handle portion 740. Sliders 746 may be near the proximalmost end of handle portion 740 and may be arranged circumferentially about handle portion 740. Tension knobs 747 may be disposed distally of a respective slider 746. Securing knob 748 may be distal of tension knobs 747. However, such an arrangement is merely exemplary.

For example, each of sliders 746 may be coupled to one of cables 706. A number of sliders 746 may correspond to a number of cables 706. Alternatively, one slider 746 may control a plurality of cables 706/hooks 708. Distal movement of slider 746 may move a corresponding cable 706 distally to deploy a hook 708 coupled to cable 706. In some examples, a distance moved by slider 746 may not correspond to a distance moved by hook 708. For example, cable 706 may be looped or folded, such that movement of slider 746 may cause hook 708 to move by a greater distal distance than that moved by slider 746. For example, each of cables 706 may be wrapped around a pulley or spool 752 (FIG. 7B, and movement of slider 746 may be configured to unwrap cable 706 from Although the term “slider” or “lever” is used herein, it will be appreciated that sliders 746 may alternatively be buttons, rockers, knobs, or any other suitable actuator. Each of hooks 708 may be deployed independently by an independent slider 746. Alternatively, a single actuator may deploy all of hooks 708 or a subset of hooks 708.

A tension knob 747 may be associated with each of cables 706. For example, device 700 may include one tension knob 747 for each of cables 706. Alternatively, tension knob 747 may control a plurality of cables 706. For example, each of cables 706 may be wrapped around a pulley or spool 752. Tension knob 747 may rotate spool 752 and/or a toothed actuator 754 (e.g., gear) so as to shorten or lengthen cables 706, thereby increasing or decreasing (i.e., adjusting) a tension on cables 706 when hooks 708 are coupled to a tissue. Each of cables 706 may be tensioned/detensioned independently from one another, using independent tension knobs 747. Alternatively, a single actuator may tension/detension all or a subset of cables 706.

Securing knob 748 may be configured to twist cables 706 or to otherwise secure cables 706 to one another or to another element of device 700 so as to maintain or secure a tension in cables 706 that was set by tension knobs 747. Securing knob 748 may be rotatable about a longitudinal axis of device 700. For example, securing knob 748 may be configured to rotate or otherwise manipulate a block 756 and/or a capsule 758 in order to twist cables 706 around one another and/or within block 756 and/or capsule 758. In examples, block 756 may include a channel/lumen for each of cables 706, such that rotation or other manipulation of block 756 may cause a knot or similar structure to be formed among cables 706 proximally and/or distally of block 756 (e.g., within capsule 758) or within block 756. Alternatively or additionally, capsule 758 may include the features disclosed above for block 756 and may be rotated or otherwise manipulated so as to secure a tension of cables 706.

Button 744 may be configured to separate cables 706 from handle portion 740. For example, button 744 may be configured to sever cables 706. For example, button 744 may cause block 756 to move distally, thereby severing cables 706. As shown in in FIG. 7C, after block 756 has moved distally, it may be received and secured within capsule 758 (e.g., via a friction fit or by mating elements of block 756 and/or capsule 758). Handle portion 740 and sheath 751 may be moved proximally relative to cables 706 and block 756/capsule 758 to remove handle portion 740 and sheath 751. For example, sheath 751 may be removed from a working channel of an insertion device. Hooks 708 may be coupled to one another by cables 706, capsule 758 and/or block 756 in the deployed configuration shown in FIG. 7C.

With reference to FIGS. 7A-7C, an operation of device 700 is described. Insertion portion 750, including sheath 751, may be inserted into a working channel of an insertion device that has been navigated to a target site. Each individual hook 708 may be used to attach to a respective target tissue. For example, an operator may slide one of sliders 746 distally along handle portion 740 to deploy a respective hook 708. The process may be repeated for a number of hooks 708 desired to be deployed. When one or more hooks 708 are in place, tensile knob(s) 747 may be actuated to provide tension to respective cables 706. Actuating tensile knobs 747 may, for example, cause rotation of toothed actuator(s) 754 and/or spools 752, as discussed above, creating tension in cables 706 and thus causing hooks 708 to pull the target tissue. As discussed above, each of hooks 708 may be deployed independently using slider(s) 746 and/or tensioned independently using knobs 747. In some examples, one or more of hooks 708 may serve to apply tension to a target tissue and one or more others of hooks 708 may serve as anchors.

After a desired number of hooks 708 have been deployed and tensioned to a desired tension, Knob 748 may then be actuated (e.g., rotated) to lock the cables 706 in a tension position, as discussed above. Button 744 may then be pressed (e.g., along or parallel to a central longitudinal axis of device 700) in order to sever cables 706. The operator may remove handle portion 740 and sheath 751, by, for example, moving handle portion 740 and sheath 751 proximally relative to cables 706, block 756, and/or capsule 758. Capsule 758 may remain coupled to proximal ends of cables 706. Cables 706 may continue to extend through the working channel of the insertion device, and capsule 758 and/or block 756 may be proximal of the proximalmost end of the working channel.

One or more other devices (e.g., knives or graspers or other devices) may be inserted in the working channel of the insertion device while cables 706 are disposed in the working channel. For example, a knife may be used to resect tissue that is engaged by at least one of hooks 708. A grasper or other device may be used to remove hooks 708 from tissues with which they are engaged. Alternatively, capsule 758 and/or block 756 may be moved proximally by an operator to remove the tissue coupled to the hook (e.g., tissue which has been severed). Capsule 758 may be grasped in order to actuate cables 706 (e.g., to move cables 706 proximally through the working channel of the insertion device).

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device comprising:
a traction hook configured for grasping tissue;
an anchor; and
a member connecting the anchor to the traction hook.

2. The medical device of claim 1, wherein the member is configured to be coupled to an external surface of a shaft of an insertion device by one or more attachment mechanisms.

3. The medical device of claim 1, further comprising:
a ring attached to the anchor, wherein
the member extends through the ring.

4. The medical device of claim 3, wherein the member is movable relative to the ring and fixedly coupled to the anchor.

5. The medical device of claim 4, wherein the anchor includes an anchor hook.

6. The medical device of claim 1, wherein the member is retractable.

7. The medical device of claim 6, wherein the anchor includes a body defining a hollow interior.

8. The medical device of claim 7, wherein the member is retractable into the hollow interior of the anchor.

9. The medical device of claim 8, wherein the member is biased into a retracted position inside the hollow interior of the anchor.

10. The medical device of claim 1, wherein the anchor includes a corkscrew.

11. The medical device of claim 1, wherein the anchor includes a weighted ball.

12. The medical device of claim 1, wherein the member is a first member, wherein the medical device includes a handle having a first actuator configured to deploy the traction hook, a second actuator configured to adjust a tension of the first member, a third actuator configured to deploy the anchor, and a fourth actuator configured to adjust a tension of a second member coupled to the anchor.

13. The medical device of claim 12, wherein the first member and the second member are coupled to one another by a capsule in a deployed configuration of the medical device.

14. The medical device of claim 12, wherein the handle further comprises a fifth actuator configured to secure the first member to the second member and thus secure a tension of the first member and the second member.

15. The medical device of claim 14, wherein the handle further comprises a sixth actuator configured to separate the first member and the second member from the handle.

16. A medical device comprising:
a handle including a plurality of first actuators, a second actuator, and a third actuator;
a sheath;
a plurality of members extending through the sheath; and
a plurality of anchoring mechanisms, each anchoring mechanism at a distal end of a respective member of the plurality of members, wherein each of the plurality of first actuators is configured to deploy at least one anchoring mechanism of the plurality of anchoring mechanisms, wherein the second actuator is configured to secure the plurality of members to one another, and wherein the third actuator is configured to separate the plurality of members from the handle and the sheath.

17. The medical device of claim 16, wherein the handle further comprises a plurality of fourth actuators, wherein each of the plurality of fourth actuators is configured to adjust a tension of a member of the plurality of members.

18. The medical device of claim 17, wherein each of the plurality of first actuators is configured to deploy the at least one anchoring mechanism independently of others of the plurality of anchoring mechanisms.

19. A medical method comprising:
attaching a traction hook to a target tissue;
placing an anchor proximate to the traction hook, wherein the anchor is connected to the traction hook via a cable, wire, or thread.

20. The method of claim 19, wherein placing the anchor proximate to the traction hook comprises attaching the anchor to a body lumen.
